(19) United States
(12) Patent Application Publication
(10) Pub. No.: US 2025/0051808 A1
(43) Pub. Date: Feb. 13, 2025

(54) BIOMETHANATION DEVICE AND ASSOCIATED METHOD

(71) Applicant: SUEZ GROUPE, PARIS LA DEFENSE CEDEX (FR)

(72) Inventors: Mathieu HADDAD, Sevres (FR); Marc CALIGARIS, Triel Sur Seine (FR); Patricia CAMACHO, Croissy Sur Seine (FR)

(73) Assignee: SUEZ INTERNATIONAL, Paris La Defense Cedex (FR)

(21) Appl. No.: 18/718,727

(22) PCT Filed: Dec. 5, 2022
(86) PCT No.: PCT/EP2022/084373

§ 371 Date: Jun. 11, 2024

(30) Foreign Application Priority Data
Dec. 13, 2021 (FR) FR2113368

(51) Int. Cl. C12P 5/02; C12M 1/00; C12M 1/107

(52) U.S. Cl. CPC C12P 5/023; C12M 21/04; C12M 23/58; C12M 29/08

(57) ABSTRACT

The invention relates to a device (10) for biomethanation of a CO- and/or H2-containing gas (8), wherein the biomethanation device is characterized in that it comprises:
a first reactor (11) comprising a cavity (12), said first reactor being configured to be placed inside a liquid bath (13) comprising at least one bacterial population, such that, when the first reactor (11) is in contact with the liquid bath (13), a biofilm is formed around the cavity (12);
an injector (7) for injecting the CO- and/or H2-containing gas (8) into the cavity (12), the biofilm being capable of carrying out a biological conversion of the CO- and/or H2-containing gas into methane;
a first CO2 injection device (14), configured to inject CO2 gas (9) into the liquid bath (13).

The present invention relates to a device for the biomethanation of gases comprising H2 and/or CO, such as syngas. The invention also relates to a device for the biomethanation of a CO- and/or H2-containing gas. The device and method of the invention are useful in the field of waste treatment, sludge treatment (particularly from wastewater treatment) and more generally in so-called “Power to Gas conversion”.

Methanation consists in reacting hydrogen (H2), carbon monoxide (CO) and carbon dioxide (CO2) to generate methane (CH4) and water (H2O).

Catalytic conversion chemical methanation methods are known. Such methods are nevertheless costly and usually involve high pressures and temperatures. These drawbacks can be avoided by using the biological route to transform H2 and CO2 and/or CO into methane at normal temperatures and pressures.

Biological methods are also known, which can be considered as biocatalysis methods using micro-organisms. Several mechanisms may be involved in the biomethanation reaction, depending on the populations of microorganisms involved. There are in particular three examples of bacterial populations that can synthesize methane (CH4) from CO or H2 via three distinct mechanisms (see Navarro et al. Front. Microbiol. 7:1188).

Population 1: Carboxydotrophic acetogens+Acetogenic methanogens.

4CO+2H2O→CH3COOH+2CO2 Reaction 1:

CH3COOH→CH4+CO2 Reaction 2:

4CO+2H2O->CH4+3CO2 Global Reaction:

Population 2: Homoacetogenic bacteria+Acetogenic methanogens or Hydrogenotrophic methanogens

4H2+2CO2->CH3COOH+2H2O Reaction 1:

CH3COOH→CH4+CO2 Reaction 2:

4H2+CO2->CH4+2H₂O Reaction 3:

4H2+CO2->CH4+2H2O Global Reaction:

Population 3: Carboxydotrophic methanogens

Global Reaction: 4CO+2H2O→CH4+3CO2

These different populations can, of course, coexist in the same reactor.

One of the well-known limitations of biomethanation methods is the low solubility of carbon monoxide (CO) and dihydrogen (H2) in water, which limits the kinetics and/or yields of biological methanation methods. Various solutions have been explored to increase the transfer of these molecules into the reaction medium.

For example, documents WO2013110186, US20160153008 and US20160230193 present methods comprising a step of circulating a pyrolysis gas (syngas) inside a digester. Such methods enable concomitant methanation of digestion reactions. However, such methods do not allow for the selection of microorganisms dedicated to methanation, and therefore limit the methanation yield.

Other solutions have been developed, using a specific unit for the methanation stage, which, via selection pressure, makes it possible to obtain a specialized population of microorganisms for methanation. In particular, WO2018234058 provides for the injection of pyrolysis gas through a membrane positioned in the methanation reactor, in order to increase the contact surface between the gas and the reaction medium, and as a result the mass transfer and yields. However, in such a system, the concentration of CO2 relative to the concentration of CO or H2 is not controlled: it results from the partial concentrations in the injected gas. Thus, the membranes are used in part to inject CO2, which is much more soluble in aqueous media than CO or H2.

There is therefore a need for improved methanation methods, particularly in terms of mass transfer of poorly soluble gases such as CO and H2, in terms of the use of membranes for injection of said poorly soluble gases, and in terms of overall conversion efficiency. Improved methods of this kind would also make it possible to reduce the size of the methanation reactor.

The invention aims to alleviate some or all of the above-mentioned problems by proposing a modular biomethanation device and method, decoupling CO2 injection from CO/H2 injection.

To this end, the invention relates to a device for biomethanation of a CO- and/or H2-containing gas, wherein the biomethanation device is characterized in that it comprises:
a first reactor comprising a cavity, said first reactor being configured to be placed inside a liquid bath comprising at least one bacterial population, so that, when the first reactor is in contact with the liquid bath, a biofilm is formed around the cavity;
an injector for injecting the CO- and/or H2-containing gas into the cavity, the biofilm being capable of carrying out a biological conversion of the CO- and/or H2-containing gas into methane;
a first CO2 injection device, configured to inject CO2 gas into the liquid bath.

Advantageously, the biomethanation device according to the invention comprises a fine bubble or microbubble or nanobubble generator comprising:
a first inlet connected to the first CO2 injection device;
a first outlet connected to the liquid bath,
the fine bubble or microbubble or nanobubble generator being configured to deliver fine bubbles or microbubbles or nanobubbles of CO2 injected at the first inlet to the first outlet.

Advantageously, the biomethanation device according to the invention comprises a second injection device connected to the liquid bath, said second injection device being designed to inject sodium bicarbonate into the liquid bath.

Advantageously, the biomethanation device according to the invention comprises a third gas injection device, preferentially CO2, configured to intermittently inject said gas in gaseous form into the liquid bath, preferentially in the form of fine bubbles or large bubbles.

Advantageously, the biomethanation device according to the invention comprises:
a device for measuring the alkalinity and/or pH of the liquid bath, and/or
a gas composition analyzer at an outlet of the first reactor, and/or
a probe for measuring CO2 dissolved in the liquid bath,
connected to the liquid bath and/or to the outlet of the first reactor and designed to determine a concentration of a control species in the liquid bath and/or at the outlet of the first reactor, the control species preferentially being CO2, H2 or methane, the biomethanation device further comprising a means of controlling the injection device based on the concentration of the control species determined.

Advantageously, the first injection device and the third injection device form a single injection device.

The invention also relates to a biomethanation installation comprising:
an anaerobic digester configured to be supplied with organic materials and to generate biogas,
a biomethanation device as described previously,
the anaerobic digester being connected to an outlet of the first reactor.

The invention also relates to a method for biomethanation of a CO- and/or H2-containing gas, wherein the biomethanation method is characterized in that it comprises the following steps:
a step of providing a first reactor placed inside a liquid bath comprising at least one bacterial population, said first reactor comprising a cavity in contact with the liquid bath, around which a biofilm is formed;
a step of injecting the CO- and/or H2-containing gas into the cavity;
a step of biological conversion of the CO- and/or H2-containing gas into methane by the biofilm;
a first step of injecting CO2 gas into the liquid bath.

Advantageously, the biomethanation method according to the invention comprises a step for generating fine bubbles or microbubbles or nanobubbles from the injected CO2.

Advantageously, the biomethanation method according to the invention comprises a step of injecting sodium bicarbonate into the liquid bath.

Advantageously, the biomethanation method according to the invention comprises a second step of injecting gas, preferentially CO2, intermittently and in gaseous form into the liquid bath, preferentially in the form of fine bubbles or large bubbles.

Advantageously, the biomethanation method according to the invention comprises:
a step for measuring the alkalinity of the liquid bath, and/or
a step for analyzing a gas composition at an outlet of the first reactor, and/or
a step for measuring CO2 dissolved in the liquid bath,
for determining a concentration of a control species in the liquid bath and/or at the outlet of the first reactor, the control species preferentially being CO2, H2 or methane,
a step for controlling the injection step based on the concentration of the control species determined.

Advantageously, the biomethanation method according to the invention comprises a step of supplying the converted methane to an anaerobic digester.

The invention will be better understood and other advantages will become apparent on reading the detailed description of an exemplary embodiment, with the description being illustrated by the attached drawings, in which:

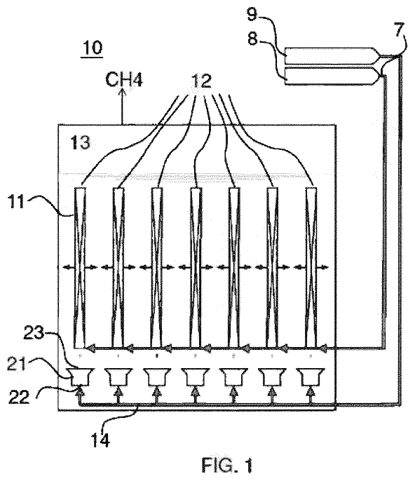

FIG. 1 schematically shows a cross sectional view of a biomethanation device according to the invention;

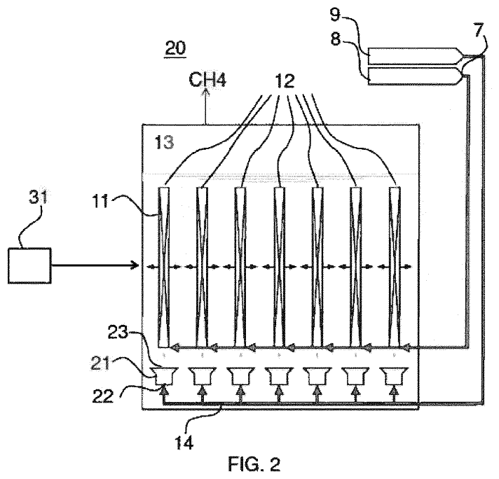

FIG. 2 schematically shows a cross sectional view of another embodiment of a biomethanation device according to the invention;

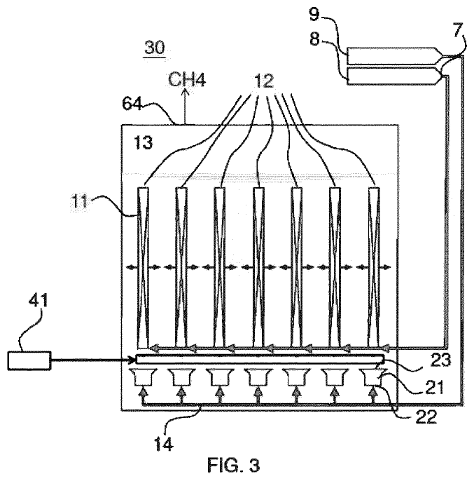

FIG. 3 schematically shows a cross sectional view of another embodiment of a biomethanation device according to the invention;

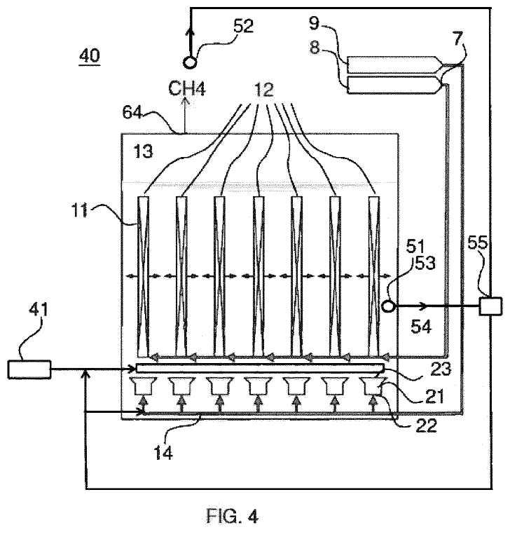

FIG. 4 schematically shows a cross sectional view of another embodiment of a biomethanation device according to the invention;

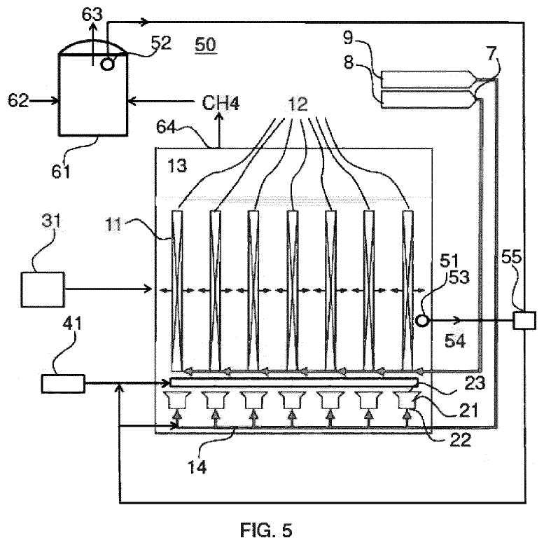

FIG. 5 schematically shows a cross sectional view of an installation according to the invention;

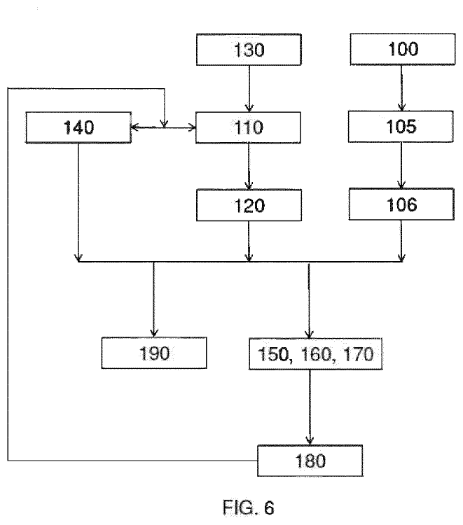

FIG. 6 schematically shows a flow chart of the steps of a biomethanation method according to the invention.

For clarity, the same elements will bear the same references across all the figures. For better visibility and understanding, elements are not always shown to scale.

FIG. 1 schematically shows a cross sectional view of a biomethanation device 10 according to the invention. The biomethanation device 10 of a CO- and/or H2-containing gas 8 comprises:
a first reactor 11 comprising a cavity 12, said first reactor being configured to be placed inside a liquid bath 13 comprising at least one bacterial population, so that, when the first reactor 11 is in contact with the liquid bath 13, a biofilm is formed around the cavity 12;
an injector 7 for injecting the CO- and/or H2-containing gas 8 into the cavity 12, the biofilm being capable of carrying out a biological conversion of the CO- and/or H2-containing gas into methane;
a first CO2 injection device 14, configured to inject CO2 gas 9 into the liquid bath 13.

The cavity 12 can be seen as a microbial biofilm growth medium into which CO- and/or H2-containing gas is injected in order to achieve transfer gas by diffusion via the medium. An example of a cavity can be a hollow-fiber membrane or two plates arranged parallel to one another and comprising hollow channels between the plates. CO- and/or H2-containing gas is injected into the channels or hollow fibers. The gas permeates through the cavity. The bacterial population attached to the cavity then gains access to the gas inside the biofilm. The gas is converted biologically. In other words, the biomethanation device aims to feed the biology to increase methane production. The CO- and/or H2-containing gas is injected into the cavity, and the biofilm grows around the cavity.

It is important to emphasize that the injector 7 injects the CO- and/or H2-containing gas 8 into the cavity, while the first injection device 14 injects CO2 gas 9 into the liquid bath 13. This therefore involves differential injection of CO2 on the one hand, and CO and/or H2 on the other. H2 and/or CO is thus injected into the lumen, that is, the hollow space defined by the cavity 12, to maximize the concentration differential (and therefore gas diffusion) on both sides of the cavity. Owing to the device of the invention, the addition of CO2 to the liquid bath is controlled to just what is needed. The CO2 injector 7 controls the quantity of CO2 introduced into the liquid bath 13. The biomethanation system according to the invention differs from the known prior art, where CO2 is often added in excess, due to uncontrolled addition at the same time as CO and/or H2.

Preferentially, but optionally, the biomethanation device according to the invention may comprise a fine bubble or microbubble or nanobubble generator 21, the generator 21 comprising:
a first inlet 22 connected to the first CO2 injection device 14;
a first outlet 23 connected to the liquid bath 13,
the fine bubble or microbubble or nanobubble generator 21 being configured to deliver fine bubbles or microbubbles or nanobubbles of CO2 injected at the first inlet 22 to the first outlet 23.

Fine bubbles refer to bubbles produced by porous or membrane diffusers (typically made of elastomer with holes 0.5 to 2 mm in diameter) and generating a plume of bubbles with an average diameter at bubble genesis of between 1 and 5 mm. Such bubbles can be generated by a porous-type generator. Microbubbles are very small bubbles on a microscopic scale. Nanobubbles are bubbles on a nanoscopic scale. Microbubbles and nanobubbles can be generated by a membrane pump generator. The type of diffuser determines the size of the bubbles obtained. The distinction between different bubble size categories corresponds to a discretization of the worlds of nanobubbles (obtained with aerators and/or flotation), microbubbles (fat/oil flotation), fine bubbles (aeration) and large/medium bubbles (aeration, stirring).

The CO- and/or H2-containing gas may also contain CO2. According to a particular embodiment, this is syngas, in particular from a pyrolysis unit. According to a particular embodiment, this is syngas from the pyrolysis of:
organic deposits with low methanogenic potential (presence of lignin, excessive dryness, etc.);
organic deposits containing methanation inhibitors or regulated compounds (micropollutants, toxic PAHs, PCBs, aromatic cycles, etc.);
Deposits with low degradation kinetics:
   long HRT (HRT stands for hydraulic retention time),
   High digester volume,
   Large footprint.

FIG. 2 schematically shows a cross sectional view of another embodiment of a biomethanation device 20 according to the invention. The biomethanation device 20 comprises a second injection device 31 connected to the liquid bath 13, said second injection device 31 being designed to inject sodium bicarbonate into the liquid bath 13. The sodium bicarbonate dissociates to produce soluble CO2. This avoids having to solubilize a gas. Alternatively, the second injection device 31 can be connected to an upstream reservoir wherein CO2 is generated from sodium bicarbonate in an acid medium. Such a non-continuous injection of excess reagent (CO2) avoids unnecessary gas recirculation.

FIG. 3 schematically shows a cross sectional view of another embodiment of a biomethanation device 30 according to the invention. The biomethanation device 30 comprises a third gas injection device 41, preferentially CO2, configured to intermittently inject said gas in gaseous form into the liquid bath 13, preferentially in the form of fine bubbles or large bubbles. The third gas injection device 41 is designed for scouring in the liquid bath 13. The gas injected in this way enables the biofilm to be at least partially swept away, thus preventing the cavity from massing. Such an injection device avoids the use of a specific pump or stirring of the liquid bath, which could damage the cavity (particularly in the case of a membrane) and/or the biofilm, and thus have a negative impact on the yield of the biological treatment. Additionally, such an injection device 41 reduces the operating costs associated with stirring. In one embodiment of the invention, the third injection device 41 can be positioned at the bottom of the cavity, and advantageously combined with the first CO2 injection device 14 as shown in FIG. 3. In another embodiment, the third injection device 41 can be positioned on the surface of the liquid bath. Finally, it is also possible to envisage two third injection devices 41, one at the bottom of the cavity and another on the surface of the liquid bath, in which case the two devices 41 may or may not be controlled individually.

In one embodiment, the first injection device 14 and the third injection device 41 form a single injection device. The injection device then combines the CO2 supply functions for the process and for scouring.

FIG. 4 schematically shows a cross sectional view of another embodiment of a biomethanation device 40 according to the invention. The biomethanation device 40 comprises:
a device 51 for measuring the alkalinity and/or pH of the liquid bath 13, and/or
a gas composition analyzer 52 at an outlet of the first reactor 11, and/or
a probe 53 for measuring CO2 dissolved in the liquid bath 13, connected to the liquid bath 13 and/or to the outlet of the first reactor 11 and designed to determine a concentration 54 of a control species in the liquid bath 13 and/or at the outlet of the first reactor 11, the control species preferentially being CO2, H2 or methane, the biomethanation device further comprising a means 55 of controlling the injection device 14, 41 based on the concentration 54 of the control species determined.

In this way, the addition of CO2 can be controlled by measuring alkalinity, in particular by direct measurement. This measurement can be carried out by an on-line sampler or analyzer (by titrimetry). One example is an on-line sequential sampling analyzer that can use a variety of automated analytical technologies to perform the analysis. Alkalinity is a measure of water's ability to neutralize acids. Alkaline compounds such as bicarbonates, carbonates and hydroxides remove hydrogen ions and lower water acidity. This is done by combining hydrogen ions to make new compounds. Total alkalinity is measured by the amount of acid required to bring the sample to a specified pH endpoint. At this pH, all alkaline compounds in the sample are “exhausted”. The result is expressed in parts per million (ppm) or milligrams per liter (mg/l) of calcium carbonate (CaCO3). It is also possible to calculate the alkalinity of the liquid bath by direct measurement of the conductivity. The addition of CO2 can also be controlled by measuring alkalinity through pH in the liquid bath (indirect measurement).

The addition of CO2 can also be controlled on the basis of gas composition, or by direct measurement of CO2 in the liquid. Such control allows the addition of just the right amount of CO2 for optimum methanation efficiency.

The composition of the gas downstream of the first reactor 11 is obtained by the analyzer 52 of a gas composition at an outlet of the first reactor 11. The analyzer 52 can, for example, be an on-line biogas analyzer.

A control species is selected in accordance with the chosen measurement method. Its concentration 54 is determined, and depending on this value, the control means 55 controls the injection of CO2 into the biomethanation device.

By way of illustration, in the case of the probe 53 for measuring CO2 dissolved in the liquid bath 13, the control species is CO2. Its concentration 54 in the liquid bath is determined by the probe. This value of CO2 concentration in the liquid bath is then compared with a CO2 concentration threshold. Above the threshold value (meaning there is too much CO2 in the system), the injection of CO2 into the liquid bath is stopped (or reduced) via the control means 55 until the concentration 54 of CO2 in the liquid bath is below the threshold value for this concentration.

The same principle can be applied for methane as a control species, downstream of the first reactor 11. The gas composition analyzer 52 downstream of the first reactor 11 determines the methane concentration 54 downstream of the first reactor 11. The control means 55 controls the injection device 14, 41 based on the methane concentration 54 determined in relation to a previously established methane concentration threshold (which may vary according to operating conditions). If the methane concentration 54 is below the threshold value, the CO2 injection device 14 and/or 41 is activated to contribute to the overall conversion of gas 8 into methane.

The invention relies on two separate and distinct sources of CO2 and CO- and/or H2-containing gas. This makes it possible to control the feed ratio between the gases.

In addition, and as a result of the inlet gas separation, the flow of injected CO2 is discontinuous. It is therefore possible to completely control its supply, and thus avoid a surplus of CO2 that would require downstream recirculation of the CO2 to achieve the necessary gas purity.

One possible implementation of the biomethanation device according to the invention is to inject the H2- and/or CO-containing gas inside the hollow material (that is, in the cavity) continuously, or intermittently, and/or in batches. Based on the overall reactions involved (some of which are mentioned in the introduction), it is possible to regulate the gas supply to the device based on the partial pressure of dihydrogen (H2) and/or carbon monoxide (CO). Injection of CO2 by the first CO2 injection device 14 is preferentially carried out discontinuously.

FIG. 5 schematically shows a cross sectional view of a biomethanation installation 50 according to the invention. The biomethanation installation 50 comprises:
an anaerobic digester 61 configured to be supplied with organic materials 62 and to generate biogas 63,
a biomethanation device as described previously,
the anaerobic digester 61 being connected to an outlet 64 of the first reactor 11. In other words, the anaerobic digester 61 is positioned downstream of the first reactor 11 and is supplied in particular by the methane converted by the biomethanation device.

In the case of this installation, the analyzer 52 may be designed to analyze a gas composition in the digester's head gas 61, and the control means 55 of the injection device 14 and/or 41 is controlled based on the concentration 54 of the control species determined in the head gas.

FIG. 6 depicted schematically shows a flow chart of the steps of a biomethanation method according to the invention.

The method for the biomethanation of the CO- and/or H2-containing gas 8 comprises the following steps:
a step 100 of providing a first reactor 11 placed inside a liquid bath 13 comprising at least one bacterial population, said first reactor 11 comprising a cavity 12 in contact with the liquid bath 13, around which a biofilm is formed;
a step 105 of injecting the CO- and/or H2-containing gas 8 into the cavity 12;
a step 106 of biological conversion of the CO- and/or H2-containing gas into methane by the biofilm;
a first step 110 of injecting CO2 gas into the liquid bath 13.

Step 105 of injecting gas 8 into the cavity can be carried out continuously, intermittently or in batches to regulate methane production.

Unless otherwise indicated, the various steps presented below are optional and can be combined in the method. In other words, they can be included individually or in groups in the biomethanation method.

In another embodiment of the invention, the biomethanation method comprises a step 120 for generating fine bubbles or microbubbles or nanobubbles from the injected CO2.

In an alternative embodiment, the biomethanation method can comprise a step 130 of injecting sodium bicarbonate into the liquid bath 13. Alternatively, step 130 can be a step for generating CO2 from sodium bicarbonate in an acid medium.

In an advantageous embodiment, the biomethanation method comprises a second step 140 of injecting gas, preferentially CO2, intermittently and in gaseous form into the liquid bath 13, preferentially in the form of fine bubbles or large bubbles. In this second step 140, CO2 gas (or biogas, methane or any other gas that does not interfere with the downstream process of upgrading the biogas produced in the digester) is injected into the liquid bath of the first reactor 11 for scouring. This second injection step prevents microorganisms from massing on the cavity. This avoids the need to stir the liquid medium (e.g. by injecting a stream of pressurized water), which could damage the membranes/biofilms. Additionally, this step 140 reduces the operating costs that would be associated with stirring the liquid bath. The gas injected in the second step 140 can be injected intermittently (e.g. once a day or once a week) via fine or coarse bubbles (e.g. the injected gas is sent via diffusers to create bubbles) to perform scouring instead of having a media agitation system. In one embodiment of the invention, the second injection step 140 can be carried out at the bottom of the cavity, advantageously combined with the first CO2 injection step 110. In another embodiment, the second injection step 140 can be carried out on the surface of the liquid bath. Finally, it is also possible to envisage two injection steps 140, one performed at the bottom of the cavity and the other at the surface of the liquid bath, which may or may not be controlled individually.

According to another embodiment of the invention, the biomethanation method according to the invention comprises:
a step 150 for measuring the alkalinity of the liquid bath 13, and/or
a step 160 for analyzing a gas composition at an outlet of the first reactor 11 (preferentially in the head gas of a digester coupled downstream of the first reactor), and/or
a step 170 for measuring CO2 dissolved in the liquid bath 13, for determining a concentration 54 of a control species in the liquid bath 13 and/or at the outlet of the first reactor 11, the control species preferentially being CO2, H2 or methane,
a step 180 for controlling the injection step 110, 140 based on the concentration 54 of the control species determined.

As explained above, the invention is based on differential injection of CO2 and H2 and/or CO with two actions on the biomethanation method.

On the one hand, the invention relates to the CO2 process wherein the addition of CO2 is controlled to the extent necessary by alkalinity (by direct or indirect measurement via pH measurement) or on the basis of the gas composition downstream of the first reactor 11, or on the basis of direct measurement of CO2 in the liquid bath 13. This control of the amount of CO2 injected into the liquid bath minimizes any downstream separation treatment. Indeed, CO2 is almost always in excess and available in the liquid medium via alkalinity. This avoids recirculating gas or having to purify it downstream.

The invention can also be applied to scouring, preferentially using CO2. There are several ways to control the need for scouring:
Alkalinity: When a drop in alkalinity is observed, this means that CO2 is accumulating (therefore CO2 that has not been consumed);
Break in slope of H2 consumption (e.g. with a flowmeter positioned at an H2/CO inlet to the cavity) due to a lack of local CO2, indicating a loss of biofilm activity that may be linked to excessive biofilm thickness;
Measurement of CH4 and/or CO2 in the biogas produced downstream in the digester indicating an increase in CO2 or with a falling CH4/CO2 ratio.

Studying these parameters makes it possible to identify when it is necessary to trigger a gas injection step for scouring.

By monitoring at least one of these parameters, it is possible to control the injection of CO2 into the liquid bath for biological conversion and the injection of gases (including CO2) for scouring.

Finally, the biomethanation method according to the invention can comprise a step 190 of supplying the converted methane to an anaerobic digester. This step enriches the digester with methane.

The invention makes it possible to increase the conversion of organic matter with low methanogenic potential but high PCI by carrying out the biomethanation of syngas, resulting from the gasification or pyrolysis of this same organic matter. The invention optimizes syngas mass transfer (CO and/or H2) and overall conversion efficiency, which is over 90%. Additionally, the proposed coupling reduces the size of the first reactor (and therefore the associated operating costs).

It will appear more generally to a person skilled in the art that various modifications can be made to the embodiments described above, in light of the teaching disclosed herein. In the following claims, the terms used should not be interpreted as limiting the claims to the embodiments disclosed in the present description, but must be interpreted to include therein all equivalents that the claims aim to cover due to their wording and which may be foreseen within the reach of a person skilled in the art based on their general knowledge.

1. A device (10, 20, 30, 40, 50) for biomethanation of a CO- and/or H2-containing gas (8), wherein the biomethanation device is characterized in that it comprises:
a first reactor (11) comprising a cavity (12), said first reactor being configured to be placed inside a liquid bath (13) comprising at least one bacterial population, so that, when the first reactor (11) is in contact with the liquid bath (13), a biofilm is formed around the cavity (12);
an injector (7) for injecting the CO- and/or H2-containing gas (8) into the cavity (12), the biofilm being capable of carrying out a biological conversion of the CO- and/or H2-containing gas into methane;
a first CO2 injection device (14), configured to inject CO2 gas (9) into the liquid bath (13).

2. The biomethanation device (10, 20, 30, 40, 50) according to claim 1, comprising a fine bubble or microbubble or nanobubble generator (21) comprising:
a first inlet (22) connected to the first CO2 injection device (14);
a first outlet (23) connected to the liquid bath (13),
the fine bubble or microbubble or nanobubble generator (21) being configured to deliver fine bubbles or microbubbles or nanobubbles of CO2 injected at the first inlet (22) to the first outlet (23).

3. The biomethanation device (20, 30, 40) according to claim 1 or 2, comprising a second injection device (31) connected to the liquid bath (13), said second injection device (31) being designed to inject sodium bicarbonate into the liquid bath (13).

4. The biomethanation device (30, 40) according to any one of claims 1 to 3, comprising a third gas injection device (41), preferentially CO2, configured to intermittently inject said gas in gaseous form into the liquid bath (13), preferentially in the form of fine bubbles or large bubbles.

5. The biomethanation device (40) according to any one of claims 1 to 4, comprising:
a device (51) for measuring the alkalinity and/or pH of the liquid bath (13), and/or
a gas composition analyzer (52) at an outlet (64) of the first reactor (11), and/or
a probe (53) for measuring CO2 dissolved in the liquid bath (13),
connected to the liquid bath (13) and/or to the outlet (64) of the first reactor (11) and designed to determine a concentration (54) of a control species in the liquid bath (13) and/or at the outlet of the first reactor (11), the control species preferentially being CO2, H2 or methane, the biomethanation device further comprising a means (55) of controlling the injection device (14, 41) based on the concentration (54) of the control species determined.

6. The biomethanation device (20, 30, 40) according to claim 4 or 5, wherein the first injection device (14) and the third injection device (41) form a single injection device.

7. A biomethanation installation (50) comprising:
an anaerobic digester (61) configured to be supplied with organic materials (62) and to generate biogas (63),
a biomethanation device (10, 20, 30, 40) according to any one of claims 1 to 6,
the anaerobic digester (61) being connected to an outlet (64) of the first reactor (11).

8. A method for biomethanation of a CO- and/or H2-containing gas (8), wherein the biomethanation method is characterized in that it comprises the following steps:
a step (100) of providing a first reactor (11) placed inside a liquid bath (13) comprising at least one bacterial population, said first reactor comprising a cavity (12) in contact with the liquid bath (13), around which a biofilm is formed;
a step (105) of injecting the CO- and/or H2-containing gas (8) into the cavity (12);
a step (106) of biological conversion of the CO- and/or H2-containing gas into methane by the biofilm;
a first step (110) of injecting CO2 gas into the liquid bath (13).

9. The biomethanation method according to claim 8, comprising a step (120) for generating fine bubbles or microbubbles or nanobubbles from the injected CO2.

10. The biomethanation method according to claim 8 or 9, comprising a step (130) of injecting sodium bicarbonate into the liquid bath (13).

11. The biomethanation method according to any one of claims 8 to 10, comprising a second step (140) of injecting gas, preferentially CO2, intermittently and in gaseous form into the liquid bath (13), preferentially in the form of fine bubbles or large bubbles.

12. The biomethanation method according to any one of claims 8 to 11, comprising:
a step (150) for measuring the alkalinity of the liquid bath (13), and/or
a step (160) for analyzing a gas composition at an outlet of the first reactor (11), and/or
a step (170) for measuring CO2 dissolved in the liquid bath (13),
for determining a concentration (54) of a control species in the liquid bath (13) and/or at the outlet of the first reactor (11), the control species preferentially being CO2, H2 or methane,
a step (180) for controlling the injection step (110, 140) based on the concentration (54) of the control species determined.

13. The biomethanation method according to any one of claims 8 to 12, comprising a step (190) of supplying the converted methane to an anaerobic digester.
